(19) United States
(12) Patent Application Publication
(10) Pub. No.: US 2026/0183580 A1
(43) Pub. Date: Jul. 2, 2026

(54) ALL-NATURAL HIGH-PERFORMANCE TRIBOELECTRIC FIBERS FOR RESPIRATORS

(71) Applicant: The Penn State Research Foundation, University Park, PA (US)

(72) Inventors: Melik DEMIREL, State College, PA (US); Khushank SINGHAL, University Park, PA (US); Ramiz BOY, University Park, PA (US); Abu Musa ABDULLAH, University Park, PA (US)

(21) Appl. No.: 19/130,064

(22) PCT Filed: Nov. 16, 2023
(86) PCT No.: PCT/US2023/080101

§ 371 Date: May 14, 2025

Related U.S. Application Data
(60) Provisional application No. 63/425,971, filed on Nov. 16, 2022.

(51) Int. Cl. A62B 23/02; A61M 16/10

(52) U.S. Cl. CPC A62B 23/02; A61M 16/105; A61M 2205/0272

(57) ABSTRACT

Provided are compositions and methods that relate to materials that contain a protein component and a polysaccharide component. The proteins include alternating repeats of crystallite-forming subsequences and amorphous subsequences. The composition are capable of forming a fiber or coating, and exhibiting a triboelectric charge, and can be used in a variety of devices.

### CROSS-REFERENCE TO RELATED APPLICATIONS

This application claims priority to U.S. provisional patent application No. 63/425,971, filed Nov. 16, 2022, the entire disclosures of each of which are incorporated herein by reference.

### STATEMENT REGARDING FEDERALLY SPONSORED RESEARCH OR DEVELOPMENT

This invention was made with government support under Grant No. W911NF-18-1-0261 awarded by the United States Army/ARO. The Government has certain rights in the invention.

### SEQUENCE LISTING

The instant application contains a Sequence Listing which is submitted in .xml format and is hereby incorporated by reference in its entirety. Said .xml file is named “074339_00256_.xml”, was created on Nov. 16, 2023, and is 2,676 bytes in size.

### FIELD

This disclosure relates to compositions comprising proteins and polysaccharides, methods of making the compositions, and devices that comprise the proteins and polysaccharides.

### BACKGROUND

Personal protective equipment, including respirators and masks, is essential for preventing the spread of infections. However, they carry financial and environmental costs due to single-use synthetic polymers. For example, the COVID-19 pandemic is estimated to generate up to 7,200 tons of single-use medical waste daily. Arguably, the most used protective equipment during the pandemic is respirators and facemasks, which are cloth, surgical, and N95 masks. As crucial as they might be in ensuring respiratory health, these masks are entirely made of synthetic polymers like polypropylene, nylon, or polyester. Unfortunately, polymer manufacturing requires nonrenewable petroleum oil, complex supply-chain demand, microfiber pollution, and a high carbon footprint. As a result, these respirators end up in landfills or marine ecosystems due to a lack of recycling, which is detrimental to the ecology. Many types of protective equipment such as respirators or facemasks uses an electrostatic charge to enhance filtration efficiency. Electrostatic charges are generated when two dissimilar materials are rubbed against each other, a well-known physical process known as the triboelectric effect. Remediating charge generation is necessary since the triboelectric effect can lead to fires, electric shock to personnel, damage to electronic equipment, and more. On the contrary, the triboelectric effect is an important solution to design problems of industrial processes, separation of materials in the recycling industry, operation of copiers and printers, etc. There is an ongoing and unmet need for improved compositions that can be used as alternatives to filters in respirators, as well as other uses for compositions formed from biodegradable materials. The disclosure is related to these needs.

### BRIEF SUMMARY

The present disclosure provides bioengineered materials that comprise tandem repeat proteins and polysaccharides. In one aspect of the disclosure the compositions can be precisely tuned to generate an enhanced triboelectric charge. The compositions are useful for, among other purposes, development of more efficient triboelectric materials and the production of advanced materials using biomanufacturing. The compositions are capable of forming a fiber or coating,

In embodiments the disclosure provides a composition comprising a polysaccharide that is optionally a cellulosic material, and one or more polypeptides which when used for combining with a cellulosic material comprise alternating repeats of crystallite-forming subsequences and amorphous subsequences. In non-limiting embodiments the compositions may be used in filters in a variety of devices. Methods of making the compositions and devices are also provided.

### BRIEF DESCRIPTION OF FIGURES

FIG. 1. Respirators and facemasks are typically made from synthetic petroleum-based polymers, which contribute significantly to plastic pollution caused by personal protective equipment. However, utilizing green technologies and renewable resources can offer a sustainable and environmentally compatible solution. By using biomanufactured respirators and masks, we can promote a circular economy.

FIG. 2a. Squid Ring Teeth (SRT) protein gene analysis followed by protein gene expression in E. coli, and protein production using a 100 L bioreactor. The biomanufactured SRT protein was blended with cellulose or cellulose triacetate to prepare the spinning dope. FIG. 2b. The solution spinning setup consists of a coagulation and two washing baths. The fibers were dried and heat-set on the heating roller before winding. Photos show bobbins of (FIG. 2c) cellulose triacetate and (FIG. 2d) cellulose fibers with pure cellulose and protein added at 10%. Fibers with higher protein content appeared slightly darker in color.

FIGS. 3a-3g. Characterization of fibers: SEM micrographs of cellulose triacetate (FIG. 3a), and cellulose fibers (FIG. 3b). Cellulose fibers have circular cross-sections and a compact microstructure as compared to triacetate fibers which contain nanopores. The lateral surface of cellulose fibers is smooth. FIG. 3c. FTIR spectra of the fibers confirm the presence of the protein and relative protein content. FIG. 3d. WAXS patterns indicate the anisotropic morphology of fibers. The intensity of cellulose peaks is high, indicating higher crystallinity. Protein peaks are not seen, FIG. 3e. Optical image of the mechanical stage for mono-filament testing. FIG. 3f. Representative stress-strain curves for fibers. Cellulose fibers are higher in strength than cellulose triacetate. FIG. 3g. The blending with protein did not significantly affect the fibers' toughness. Cellulose fibers are stiffer than triacetate fibers.

FIGS. 4a-4e. Triboelectric properties of protein/cellulosic fibers: FIG. 4a. Schematic showing the construction of fiber-based nanogenerators. The top layer containing fibers is finger-tapped with a nominal force. FIG. 4b. The mechanisms of contact electrification and electrostatic induction. FIG. 4c. As recorded, triboelectric outputs for the bioengineered SRT protein film, with maximum values noted. The maximum normalized open-circuit voltage and short-circuit current are shown for both single (FIG. 4d) and two-electrode (FIG. 4e) configurations. Cellulose fibers outperformed triacetate fibers. Protein functionalization further increased the electrical output for both material types. Protein-coated pure cellulose fibers represent the theoretical maxima of the triboelectric output.

FIG. 5. The Fig. of merit for triboelectric properties: Max norm. voltage and current in various fiber-based materials. The results are segregated into categories, namely, polymer, cellulose, and protein-based materials.

FIGS. 6a-6b. SEM micrographs of cellulose triacetate (FIG. 6a) and cellulose (FIG. 6b) fibers with 1% and 5% protein content. 10% is shown in the FIG. 3a.

FIGS. 7a-7b. SEM micrographs of as-spun cellulose fibers with no protein (FIG. 7a), and 10% protein (FIG. 7b) content. As-spun fibers are collected in the coagulation bath prior to any drawing.

FIGS. 8a-8b. FTIR of cellulose triacetate (FIG. 8a) and cellulose fibers (FIG. 8b) with all protein contents are shown. The spectra of the solvents are included as well.

FIGS. 9a-9b. Deconvolution of Amide I bands of cellulose (FIG. 9a) and CTA (FIG. 9b) fibers with 10% protein content. The fractions of different secondary structures are tabulated. A significant fraction of the protein is in random coil and helix morphology. The error of fit for the peak at ˜1701 cm⁻¹ for CTA is greater due to the overlapping carbonyl peak, which is likely resulting in overestimation of β-sheet content.

FIGS. 10a-10b. Representative and non-limiting illustration of the construction of the bioengineered protein film-based single electrode (FIG. 10a) and two-electrode (FIG. 10b) triboelectric nanogenerators.

FIGS. 11a-11d. Triboelectric output of fiber-based devices in single (FIGS. 11a, 11b) and two-electrode (FIGS. 11c, 11d) configurations. V_{OC} and I_{SC} denote open-circuit voltage and short-circuit current, respectively. The results for protein-coated cellulose fibers represent maximum attainable output through blending with protein.

FIG. 12. Representative and non-limiting schematic for the triboelectric series showing the relative positions of the materials used an embodiment of this disclosure.

FIG. 13. SEM micrographs of the protein-coated cellulose fibers. Protein deposits can be noticed on the fiber surface.

FIGS. 14a-14b. SEM micrographs showing the (FIG. 14a) cross-section and (FIG. 14b) lateral surface of wood (high molecular weight) cellulose-protein composite fibers. A diameter of ˜15 μm was attained.

FIGS. 15a-15c. Protein recovery from blend fibers. FIG. 15a. The protein dissolved in DMSO, after leaching out of the fibers, can be precipitated using water. FIG. 15b. FTIR spectra of the residue from DMSO treatment and the treated fibers show protein presence and reduced protein content, resp. FIG. 15c. SEM micrographs showing the change in fiber microstructure after DMSO treatment. The diameter of the fibers increased by about 2 μm, indicating increased porosity.

### DETAILED DESCRIPTION

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value encompasses variations of +/−10%, +/−5%, or +/−1%.

This disclosure includes every amino acid sequence described herein and all nucleotide sequences encoding the amino acid sequences. Polynucleotide and amino acid sequences having from 80-99% similarity, inclusive, and including and all numbers and ranges of numbers there between, with the sequences provided here are included in the invention.

The disclosure includes all methods of making the described compositions. The disclosure includes all properties of the described compositions, including but not necessarily limited to triboelectric properties, voltages, sizes, and size ranges of sizes (e.g., diameter, length, and wight), thickness, tensile strength, toughness, resistance to deformation, elastic modulus, and yield strength.

In one embodiment, the disclosure provides a composition comprising a polysaccharide that is optionally a cellulosic material, and one or more polypeptides which when used for combining with a cellulosic material comprise alternating repeats of crystallite-forming subsequences and amorphous subsequences. In embodiments the composition is capable of exhibiting a triboelectric charge, as illustrated in the figures and description below.

Any described composition may comprise or consist of the described protein and polysaccharide components. The described compositions are tunable, meaning their properties can be changed by altering the protein and/or polysaccharide components, such as their respective weights of the composition, and/or changing the length/weight of the protein component, including but not necessarily limited to changing the repeat number.

In non-limiting embodiments the crystallite-forming subsequences of a protein component comprise β-sheets. In non-limiting embodiments the crystallite-forming subsequences comprise 2 to 20, or more repeats. In non-limiting embodiments the amorphous subsequence comprises each comprise 10 to 60 amino acids. In a non-limiting embodiment, a protein component of a described material consists of 11 repeating units. In an embodiment, when a described protein is combined with a polysaccharide such as a type of cellulosic material, the protein includes random coil and α-helix structures. Suitable polypeptides comprising crystallite-forming subsequences and amorphous subsequences are described in, for example, PCT publication WO 2014/160131, U.S. Pat. Nos. 9,663,658, 10,253,144, PCT publication WO 2016/172716, U.S. Pat. Nos. 9,765,121, 10,047,127, 10,246,493, PCT publication PCT/US17/66380, and U.S. Pat. No. 11,739,164, the entire disclosures of each of which are incorporated herein by reference. In a non-limiting embodiment, the crystallite-forming subsequences comprise or consist of the sequence AAASVSTVHHP (SEQ ID NO:1). In a non-limiting embodiment, the amorphous subsequences comprise or consist of the sequence YGYGGLYGGLYGGLGYGP (SEQ ID NO:2).

The polysaccharide component of a described material may be any suitable polysaccharide. In embodiments the polysaccharide is a linear polysaccharide. In an embodiment the polysaccharide is chitin, alginate, hyaluronan and poly-N-acetylglucosamine (PNAG) or Pullulan. In an embodiment the polysaccharide is any type of cellulose. In this regard, cellulose has been used as an energy source through direct combustion or utilized by the pulp and paper industry if the cellulose has a high molecular weight (for example, in wood). The present disclosure in certain aspects provide novel processing methods that retain the molecular structure of cellulose such that higher-value materials can be achieved. High-strength cellulosic fibers (i.e., 925 MPa or 61.5 cN/tex⁸) are used in non-limiting embodiments of the disclosure and thus can replace conventional synthetic fibers (e.g., polyester). In embodiments, the cellulose is a naturally occurring cellulose, or is any of methylcellulose, thiolated cellulose, ethylcellulose hydroxypropyl methylcellulose, cellulose acetate, or cellulose triacetate.

As discussed further herein, in non-limiting approaches the present disclosure provides designed and engineered squid ring teeth (SRT) proteins with regenerated cellulose to generate an enhanced triboelectric charge material. The described functional fibers and films from SRT proteins have applications in many areas of engineered materials, including soft photonics and advanced materials. In embodiments a described composition is provided as a filament or fiber. In embodiments the composition is used to produce a filter. In embodiments the filter may be a component a component of filtration mask, a respirator, a powered air purification device (PAPD), a ventilator, a gas turbine or compressor air intake filter, a High-Efficiency Particulate Air or (HEPA) filter, or any other device that uses a filter designed to collect particles from air. In non-limiting examples the filter may be a component of a disposable or reusable cartridge. In embodiments, such as for a face covering or respirator, the composition may exhibit breathability characteristics. Passing air through a filter produces a triboelectric effect. In embodiments, a described filter may collect suspended dust and fine particles in atmospheric air, nano-aerosols, volatile organic compounds, and/or submicron particles during at least the initial use of the filter. Thus, in an aspect, the disclosure includes a method comprising passing air through a filter, wherein a triboelectric charge is formed in the filter such that particulate matter in the air is trapped on or within the filter.

In certain embodiments, polypeptides in a described composition comprise approximately 1-60% of the weight of the fiber, inclusive, and including all numbers and ranges of numbers between 1-60%. In embodiments, a described fiber has a diameter of 10-40 microns, inclusive, and including all numbers and ranges of numbers between 10-40 microns. In an embodiment the disclosure provides protein-coated cellulose fibers.

In embodiments the disclosure comprises a method of making a described composition. In one approach, the method comprises solution spinning a composition comprising a described cellulosic material and one or more described polypeptides to obtain a fiber comprising or consisting essentially of the cellulosic material and the one or more polypeptides.

In an embodiment, a described composition is used as a coating. In a non-limiting demonstration, a cellulosic material is coated with a described protein, such as by using a dip coating method. This can be used to produce a protein film on the surface of the fibers.

In an embodiment, the disclosure provides a system that comprises a conduit in fluid communication with a described filter and is configured such that air passing through the filter immobilizes particular matter in the air on or within the filter.

The following Examples are intended to illustrate but not limit the disclosure. The Examples in part describe materials that are engineered to enhance their physical properties using proteins and polysaccharides, wherein the described proteins can acquire high electrostatic charges based on the charged amino acids, and thereby demonstrate triboelectric response.

### Example 1

This Example provides materials and methods used to obtain the described results.

Materials: Extra pure microcrystalline cellulose and cellulose triacetate were used as received from ThermoScientific Chemicals. 1-Ethyl-3-methylimidazolium acetate (EmimAc), with >95% purity, was purchased from Iolitec Inc., and Oakwood Chemical provided dimethyl sulfoxide (DMSO, 99.9%).

Bioengineered SRT protein synthesis: The proteins were engineered using protein expression, gene sequencing, and protein design according to a previously described protocol (Jung, H. et al. Molecular tandem repeat strategy for elucidating mechanical properties of high-strength proteins. Proc. Natl. Acad. Sci. 113, 6478-6483 (2016)), from which the disclosure is incorporated herein by reference. The DNA sequences were verified in plasmids and then transferred to E. coli (BL21 strain with pet14b plasmid). After colony inoculation and fermentation, cells were collected and grown based on the earlier protocols. Finally, the fermentation biomass is processed to acquire purified proteins.

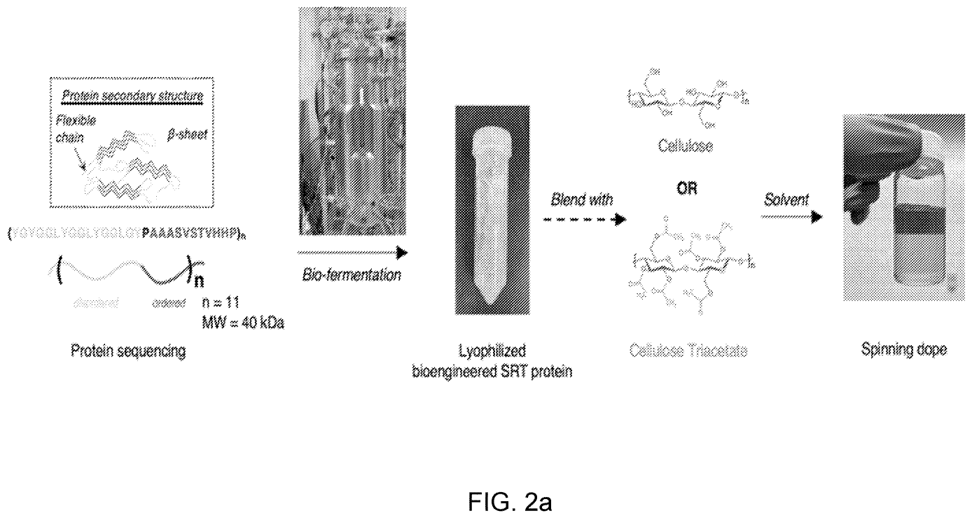

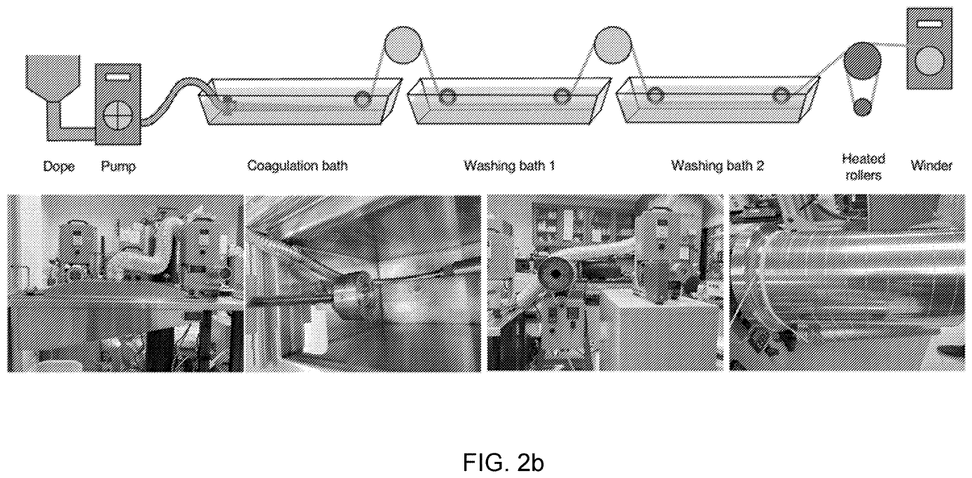

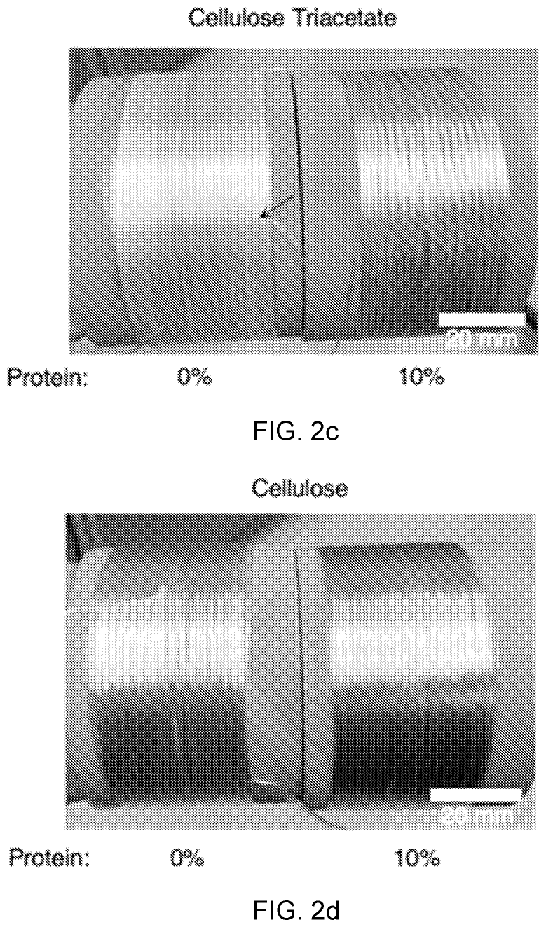

Solution spinning: The wet spinning process was continuous, resulting in filaments without breakages. Fibers were produced using a laboratory-scale line (Alex James & Associates Inc.) with components shown in FIG. 2b. Experimental methods like syringe-wet spinning do not accurately represent traditional solution spinning, as the fiber properties vary throughout their length. Short, thick fibers cannot represent textile fibers' mechanical properties. Cellulose and cellulose triacetate solutions were spun with SRT protein at weight fractions of 0%, 1%, 5%, and 10%. EmimAc and DMSO mixture was used as a dope solvent at a 1:1 ratio. The dope solid fraction of cellulose and triacetate solutions was 16% and 13% w/v, respectively. Homogeneous solutions were obtained after stirring for 3 hours at 65° C. High temperatures were avoided at all stages of spinning to prevent biopolymer degradation. A spinneret made of stainless steel with 100 orifices, each with a diameter of 100 μm, was utilized. The pump was maintained at a temperature range of 65-80° C., and its speed was adjusted to ensure the velocity of the extruded fibers was between 5-6 m/min. Fibers were extruded in a coagulation bath of DI water at room temperature, followed by two washing baths maintained at 75° C. and 60° C., respectively. The fibers were dried and annealed on a heated roller at 85° C.

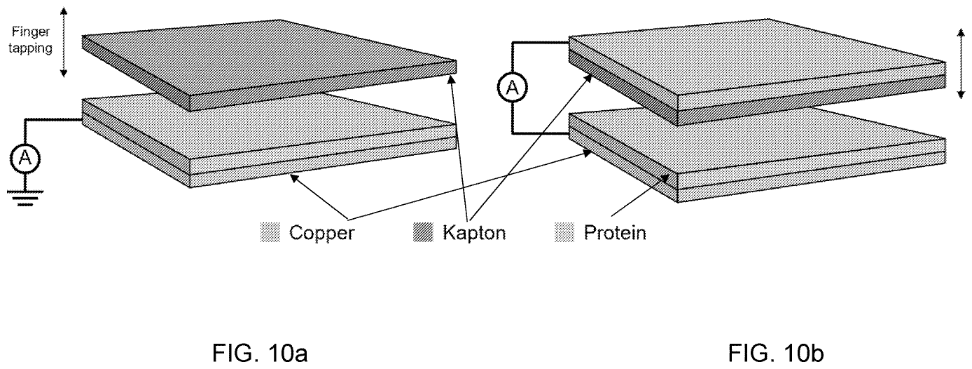

Characterization of triboelectric properties of protein film and fibers: Each multifilament was cut into staple fibers and deposited onto Kapton tapes (CGS, 0.0635 mm thickness) of 2×1.2 cm². The apparent surface areas of the fibers in all devices are listed in Table S2. The protein thin film was deposited onto a 3×3 cm2 Cu sheet using a 500 μl solution containing 10% w/v protein in HFIP solution. Once the film was formed, it was rinsed with ultrapure water and left to dry in the ambient air. Kapton films were attached to copper sheets to create the bottom layers for triboelectric measurements, as shown in FIG. 10. Polyethylene foam sheets, which were 0.5 cm thick, were used as insulation between the upper and lower layers. The voltage and current were measured using Siglent SDS 1104X-E oscilloscope and MetroOhm Autolab Pgstat128n Potentiostat, respectively. The experiment involved tapping fingers at a frequency of 2 Hz while referencing a metronome. The detailed mechanism of functioning of single and two-electrode triboelectric measurements is discussed elsewhere¹⁸. Throughout the text, the terms ‘voltage’ and ‘current’ refer specifically to open-circuit voltage (VOC) and short-circuit current (ISC), respectively.

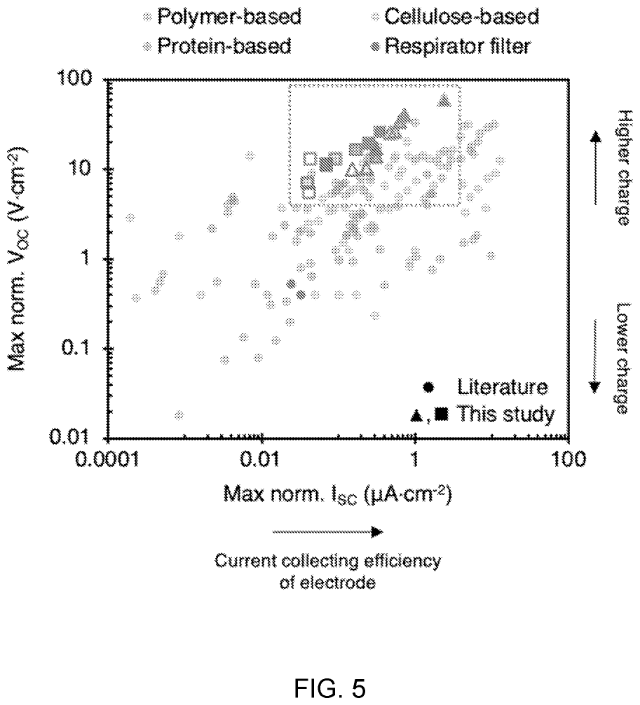

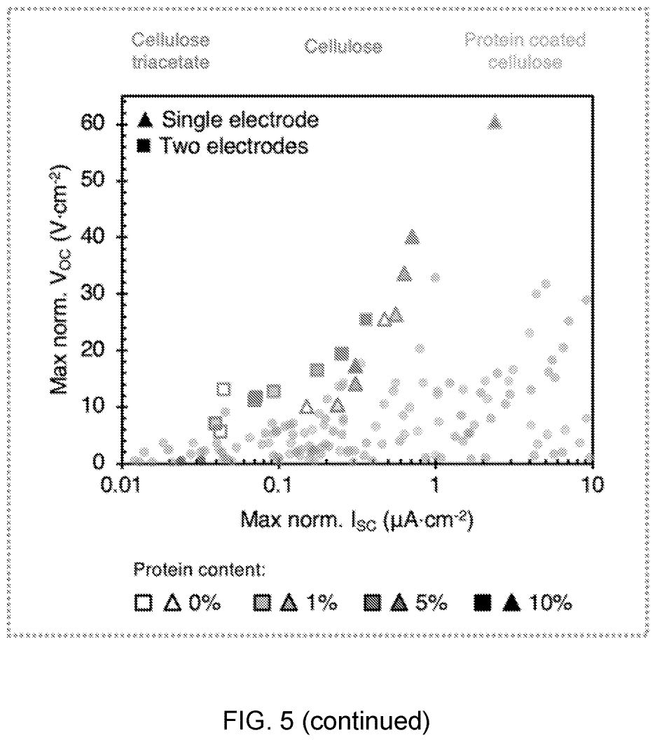

Fibers have a larger surface area and facilitate electric charge transfer compared to films. It is desirable to exclude the geometric influence on the output of devices when comparing materials based on their intrinsic triboelectric performance. We implemented several precautions to ensure accurate comparisons between our fibers and other materials. Initially, we standardized the electrical outputs by factoring in the planar area of the devices' triboelectric layer and electrodes. Additionally, we limited our analysis to materials with a fibrous morphology. Lastly, we refrained from examining devices that contained materials polarized by high-voltage treatment. In FIG. 5, devices with all possible combinations of test protocols (single or two-electrode) and structural morphologies of fibers are included.

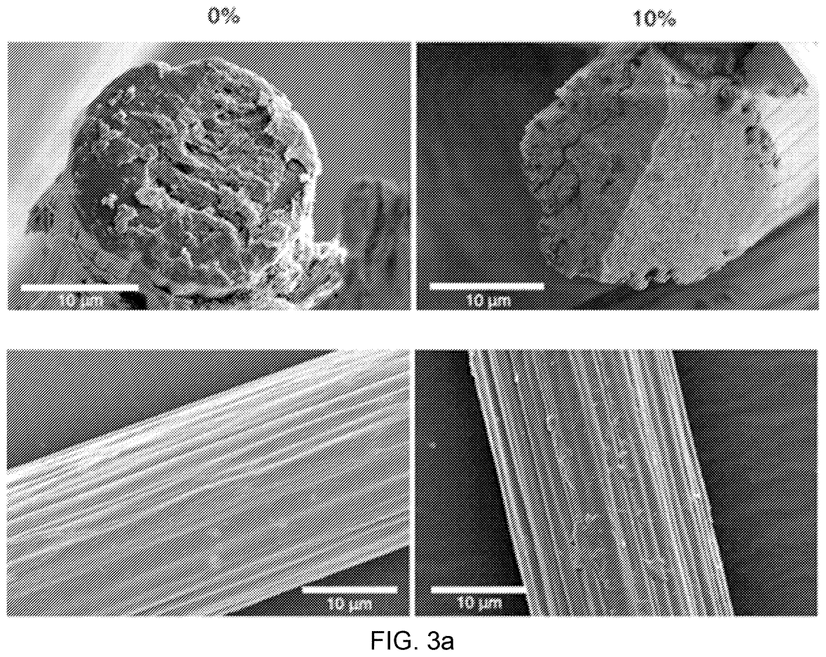

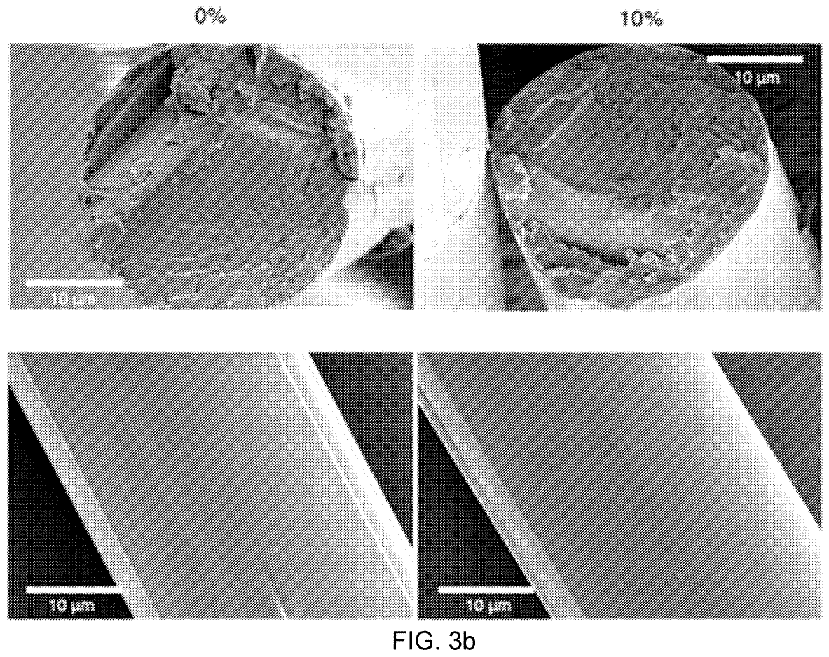

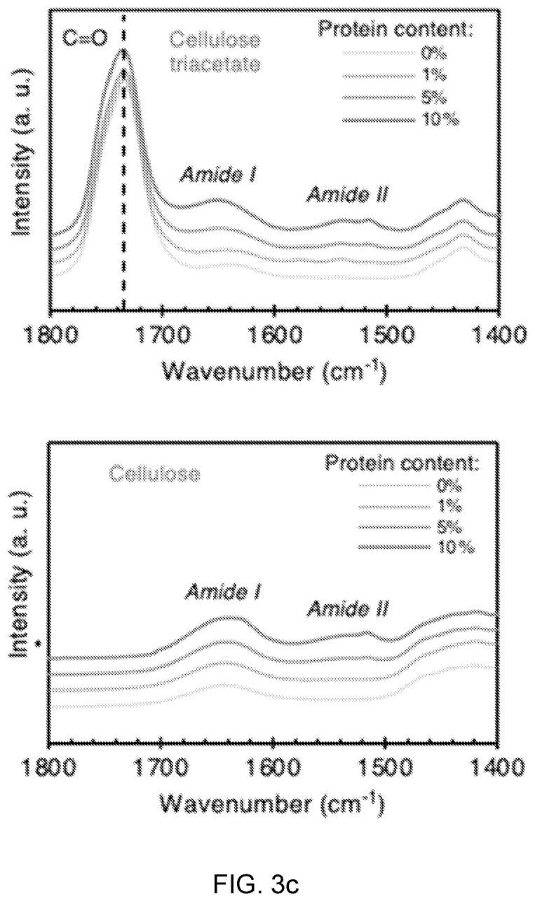

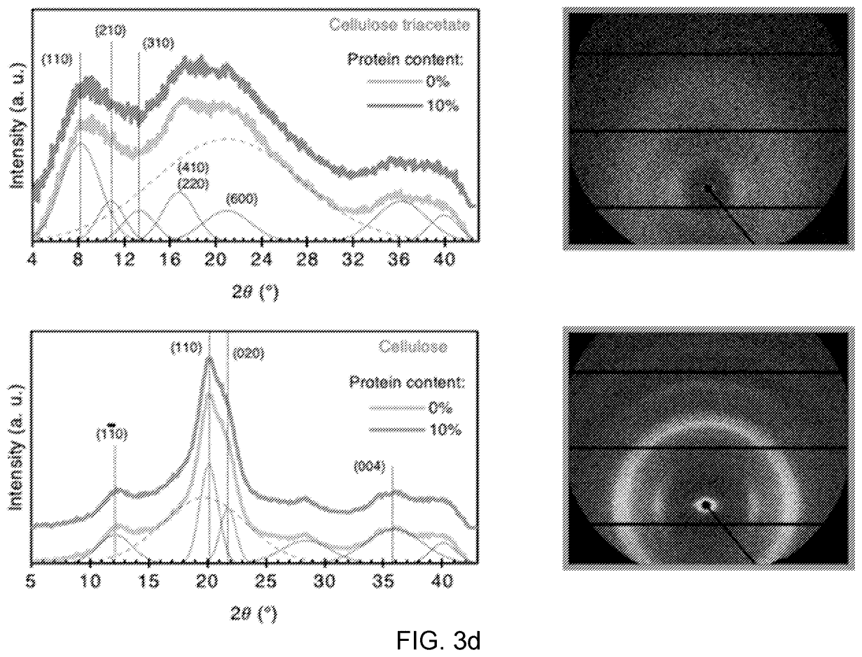

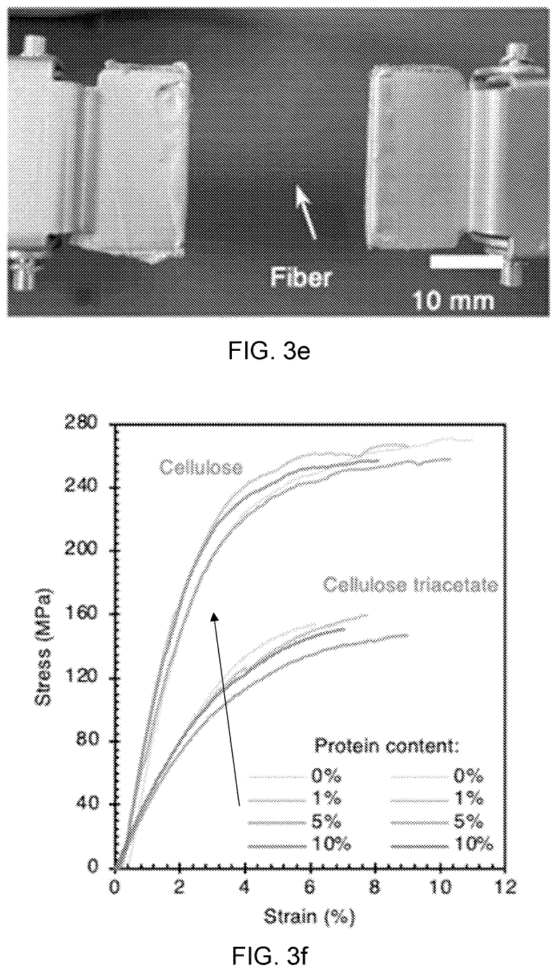

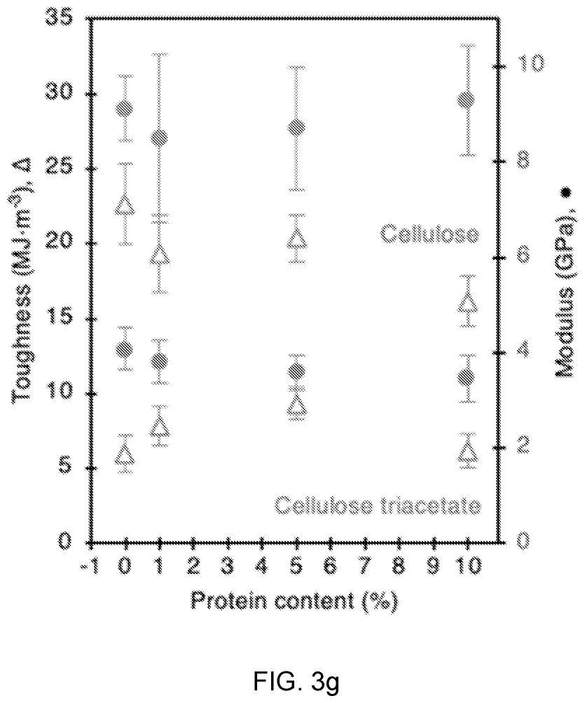

Other characterization: ATR-FTIR spectroscopy was carried out on Bruker Vertex 70 equipped with a liquid nitrogen-cooled MCT detector using a diamond crystal accessory. The spectrum was collected at a resolution of 4 cm^{-1,} and 256 or 512 scans were coadded. WAXS analysis of fibers was performed on a Xenocs Xeuss 2.0 system, operating at 50 kV and 0.6 mA current. The fibers were tested mechanically on an MTS Criterion load frame with 10 N load-cell and spring-loaded clamp-type grips, as shown in FIG. 3e. The tensile testing was carried out by the standard ASTM D3822-07, and a gauge length of 25 mm was maintained across all samples. SEM micrographs were acquired on ThermoScientific Verios G4 FESEM. Before imaging the cross-sections, the fibers were fractured using liquid nitrogen and then sputter-coated with iridium.

### Example 2

This Example provides results obtained from using the materials and methods described in Example 1, and discussion of the results.

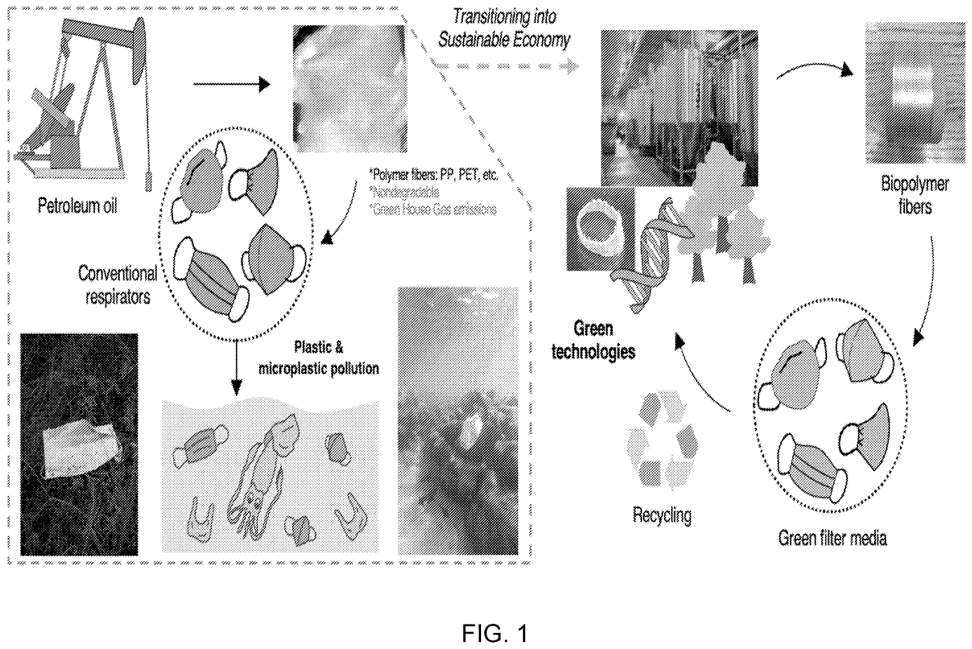

Polyester fibers have a high triboelectric value, making them a popular choice for synthetic materials that are affordable to manufacture. As a result, they are widely used in various products, such as face masks and respirators, as shown in FIG. 1. However, polyester is an oil-based plastic that does not biodegrade and takes several decades to decompose in landfills. Thus, in an embodiment, a described composition may be free of any polyester. Additionally, microplastics that originate from polyester goods pollute rivers and oceans, causing damage to plankton populations. Thus, in an embodiment, a described composition may be free of any plastic. Due to their charged amino acids, proteins can become highly electrostatic and exhibit triboelectric response, serving as a natural alternative to polyester. Proteins enhance the material's triboelectric capabilities and mechanical and surface properties. Proteins have many advantages as triboelectric materials, including eco-friendliness and customizable amino acid compositions for a higher electrostatic charge.

A segmented copolymer protein was designed using the amino acid sequence of crystal-forming (AAASVSTVHHP (SEQ ID NO1)) and amorphous (YGYGGLYGGLYGGLGYGP (SEX ID NO:2) sections, used to illustrate one non-limiting embodiment. The alanine-rich segments undergo β-sheet formation while the glycine-rich elements interconnect these nanocrystals, forming the protein matrix's flexible regions. The protein was manufactured in E. coli BL-21 strain using a 100 L fermenter through heterologous expression, as shown in FIG. 2a. Bioengineered SRT proteins have tunable chain lengths. The elastic modulus and the yield strength of these samples are similar; but their toughness and extensibility (i.e., 2%, 4.5%, and 15% for n=4, 7, and 11, respectively, where ‘n’ denotes the repeat number) increase as a function of polypeptide molecular weight. Hence, we selected as a non-limiting example the protein variant with 11 repeating units (i.e., the molecular weight of 39.9 kDa) with the highest toughness and extensibility.

### Structural and Mechanical Characterization

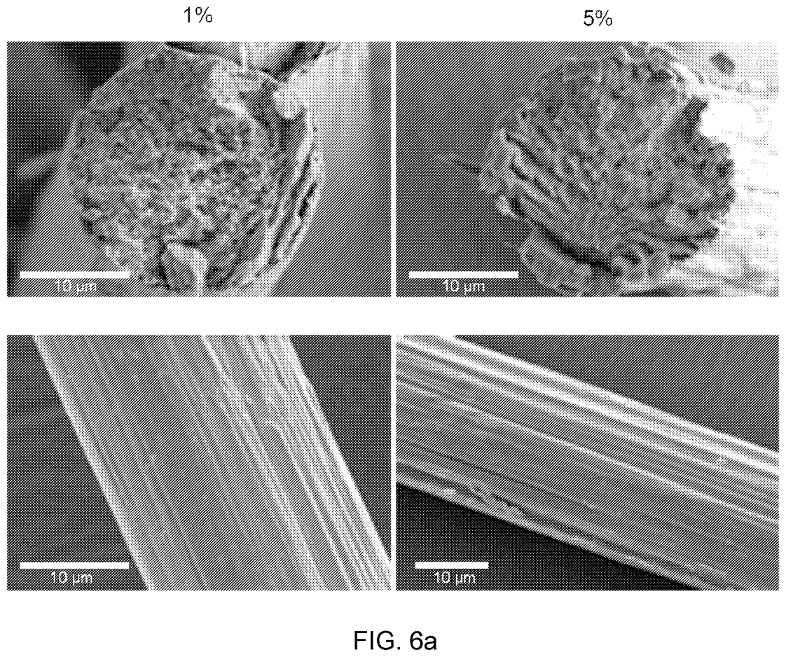

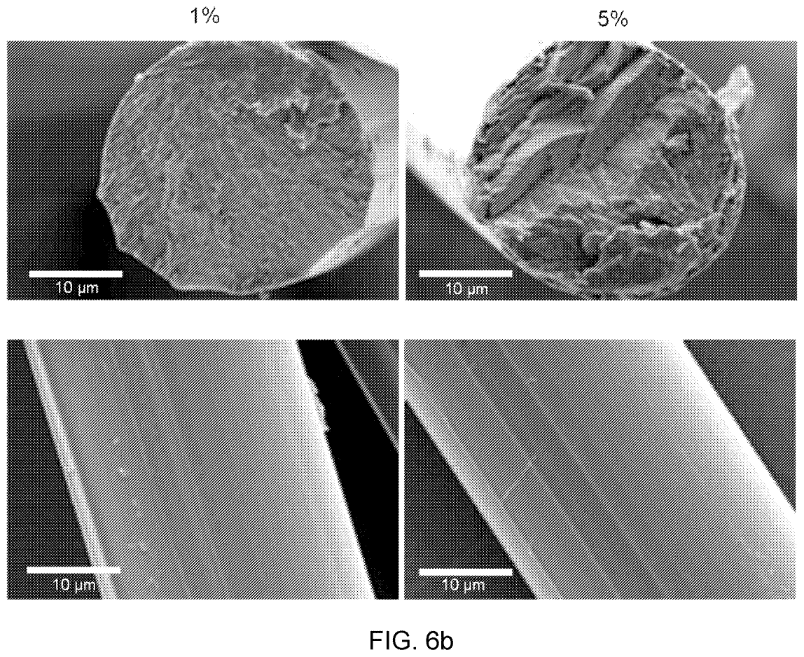

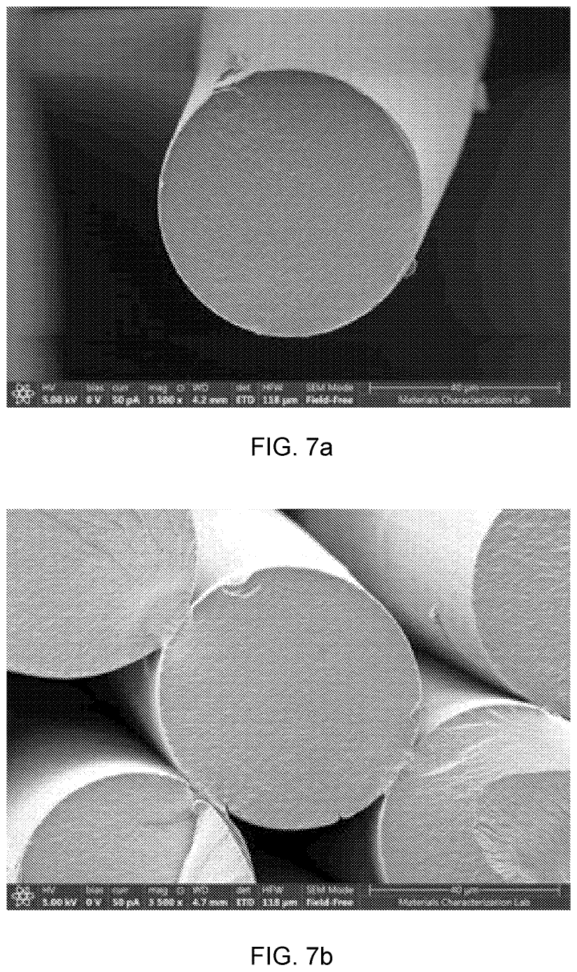

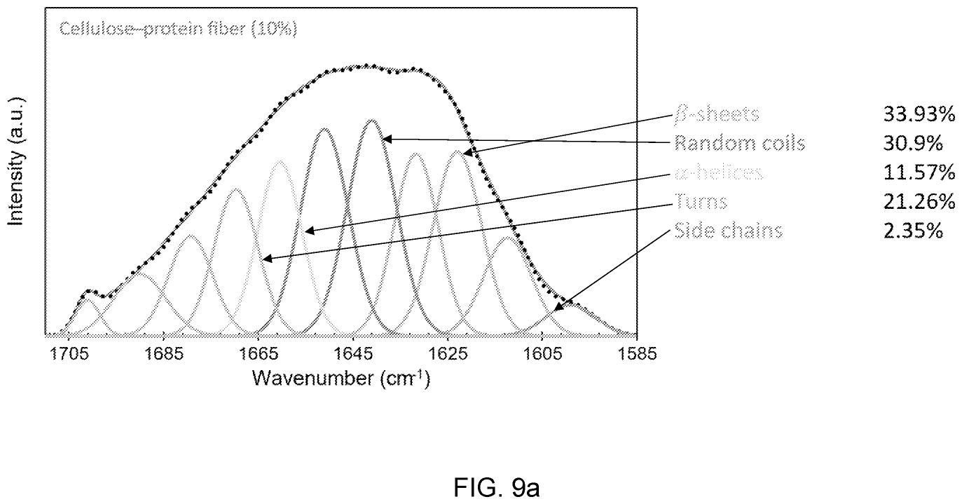

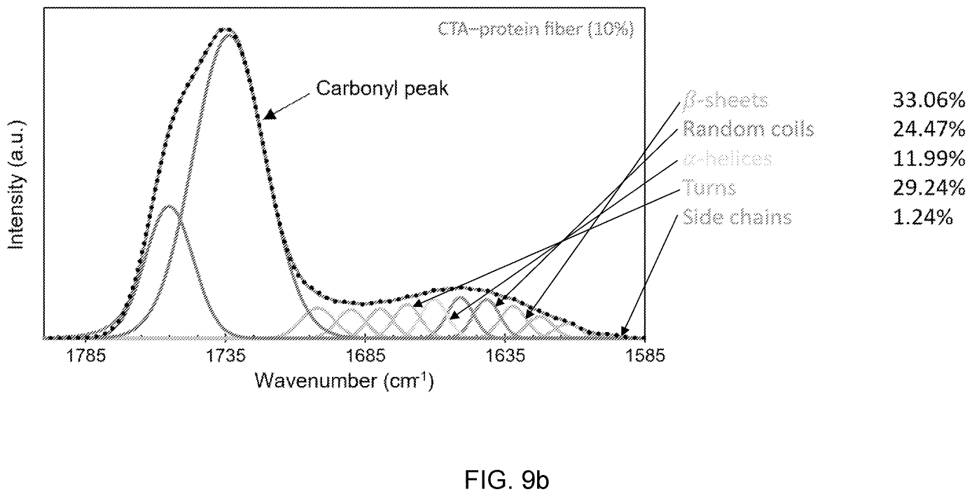

We studied fibers' chemical composition, morphology, and mechanical properties using spectroscopic and diffraction techniques and tensile tests following ASTM standards. FIGS. 3a, 3b, and FIG. 6 show the scanning electron microscope (SEM) micrographs of the fiber cross-sections and lateral surfaces. The diameter of the drawn cellulose triacetate fibers was 21.5±1.2 m, while the cellulose fibers had a diameter of 28.6±0.6 m. Based on the diameter of the as-spun cellulose fibers (56.3±1.6 m), a draw ratio of 2.6 and 2.0 was calculated for the two types of fibers, respectively (refer to FIG. 7). The Amide I and II (FIG. 3c) bands of Fourier transform infrared spectroscopy (FTIR) confirmed the secondary structure formation of the protein. The β-glycosidic linkages (895 cm⁻¹) and carbonyl (1730 cm⁻¹) bands of the FTIR spectra confirmed the cellulose and cellulose triacetate composition of the fibers, respectively (FIG. 7). FTIR deconvolution showed that SRT, when mixed with cellulose, has a notable presence of random coil and α-helix structures. This significantly contributes to its effective dispersion within the fiber matrix as shown in FIG. 9.

The wide-angle X-ray scattering (WAXS) patterns indicate anisotropic fiber morphology (FIG. 3d), which is expected for drawn fibers. Drawing also induces constituent fibrils to align along the fiber axis, which contributes to fiber strength². Cellulose and cellulose triacetate exhibit various allomorphs, among which cellulose-II has is in the described fibers. The cellulose peaks (2θ=20.1°, 21.5°) were more pronounced than those of triacetate, indicating higher crystallinity of cellulose fibers. The crystallinity of cellulose fibers was estimated to be 56.4%, compared to 44.2% for triacetate. Cellulose triacetate has a low degree of crystallization because of the bulky acetate groups on each monomer and increased inter-chain separation. WAXS patterns of pure cellulose and protein blend fibers are very similar. The fibers made from regenerated cellulose have a complicated and hierarchical arrangement. This arrangement includes basic units called fibrils, which consist of both crystalline and amorphous segments with small spaces between them. According to Zahra. et al²², the structure and organization of these cellulose fibrils mixed with 10% keratin remain unchanged, indicating that the hierarchical structure is still present within the network. Hence, protein domains in the fibers do not contribute to X-ray scattering. It can be inferred that SRT remains uniformly dispersed within the cellulose matrix and does not aggregate (FIG. 3). In contrast to previous studies, the biomanufactured SRT does not leach out into the coagulation bath during spinning of cellulose protein blends.

The representative tensile test curves of fibers are shown in FIG. 3f. The cellulose blend fibers show higher breaking strength (256.1±2.1 to 265.8±3.9 MPa) than cellulose triacetate (136.5±12.1 to 152.8±6.8 MPa). In addition, cellulose fibers are approximately 2.4 times stiffer than triacetate fibers (see FIG. 3g). Cellulose fibers exhibit superior properties due to inter- and intramolecular hydrogen bonding, higher solid content in the dope, and fibrillation. Since SRT proteins only make up 10% of the blend fibers, their mechanical properties do not significantly affect the overall mechanical properties of the blend. As a result, the properties of both cellulose and cellulose triacetate fibers remain unchanged.

### Triboelectric Properties of Regenerated Fibers

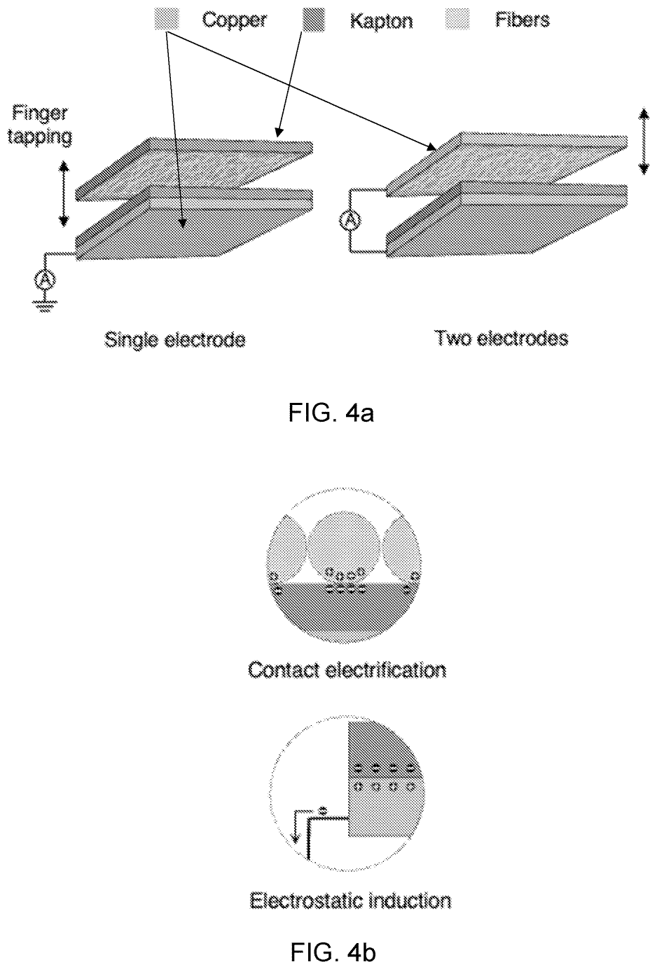

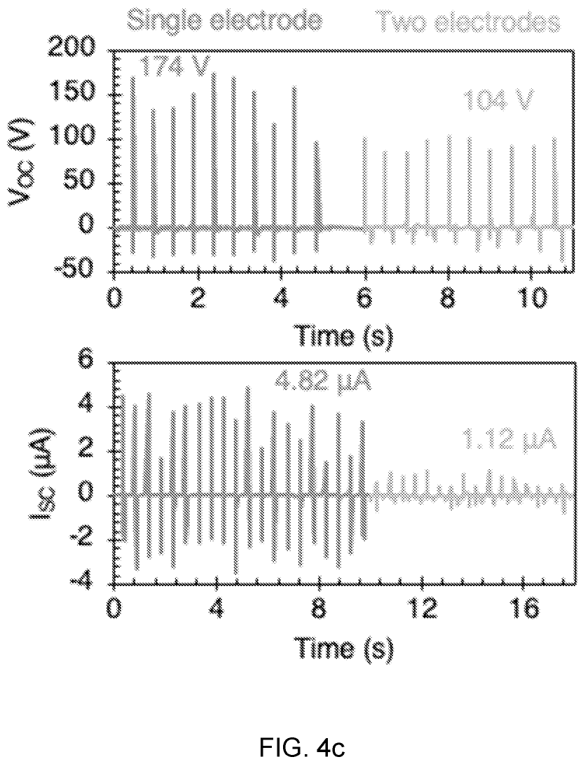

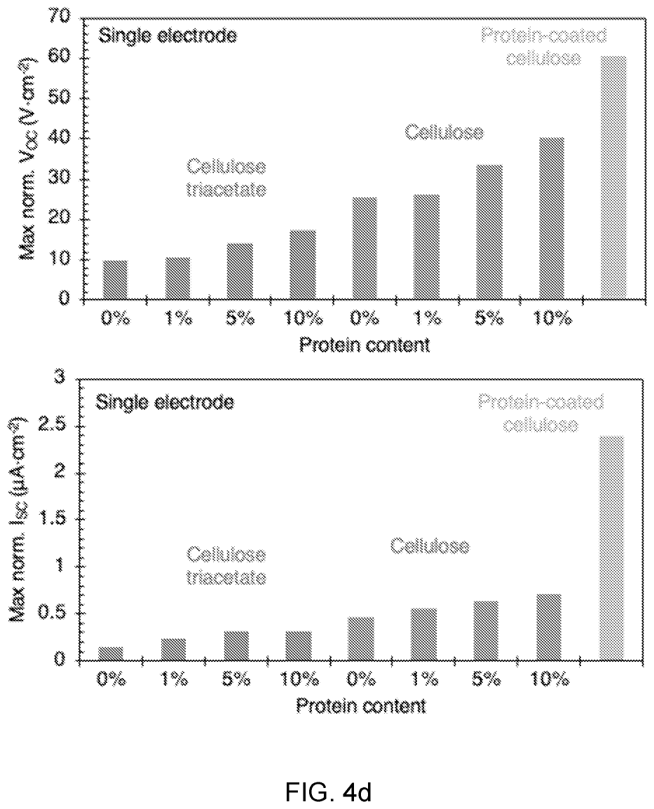

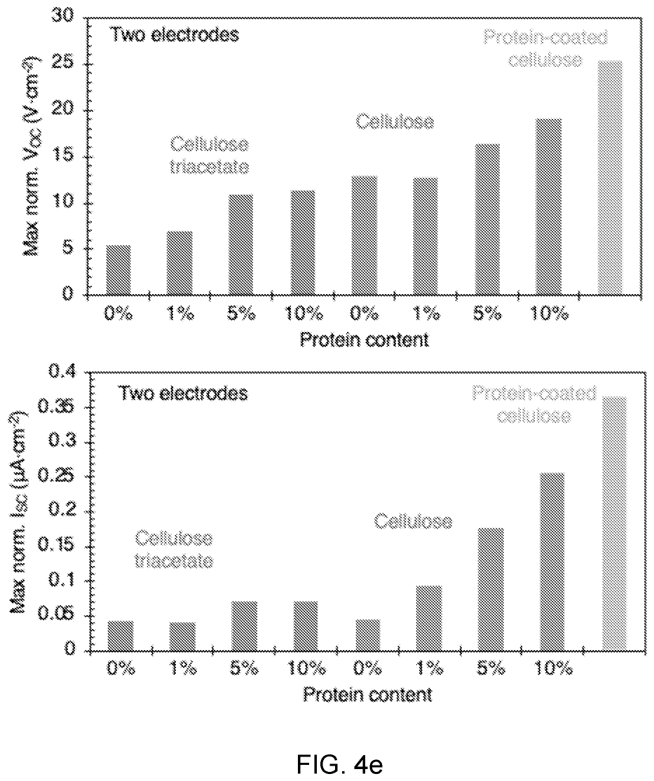

To analyze the triboelectric properties of our blend fibers, we performed electrical measurements in both single-electrode and two-electrode configurations, as depicted in FIG. 4a and S5, respectively. This allowed us to examine how alternating potentials and currents are generated through contact electrification and electrostatic induction, as shown in FIG. 4b. This setup imitates the charging process of a mask or respirator while breathing. It should be noted that the use of sliding mode with fiber-carrying electrodes can cause fiber abrasion and piling up. Thus, alternative operational modes to avoid these issues are encompassed by the disclosure.

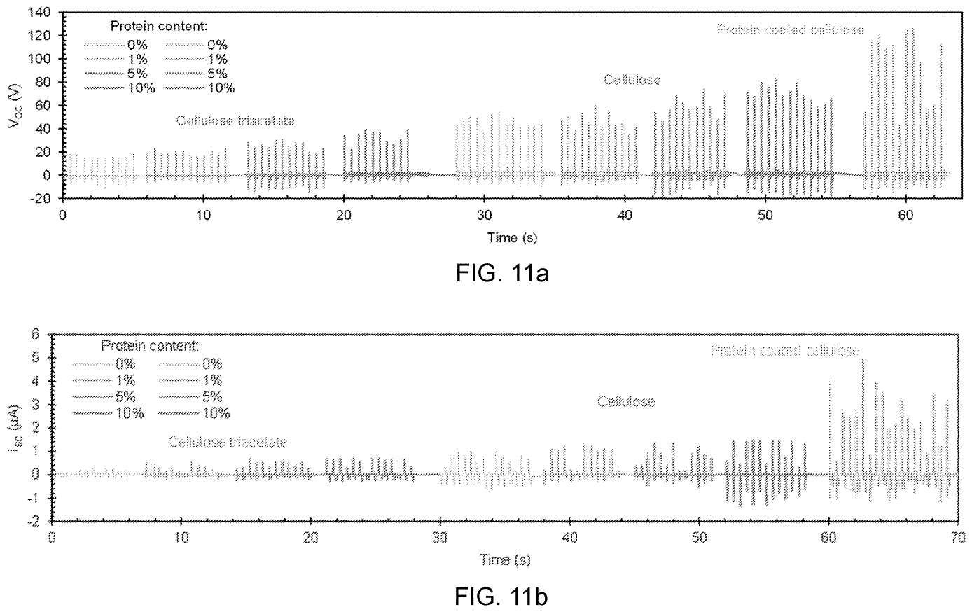

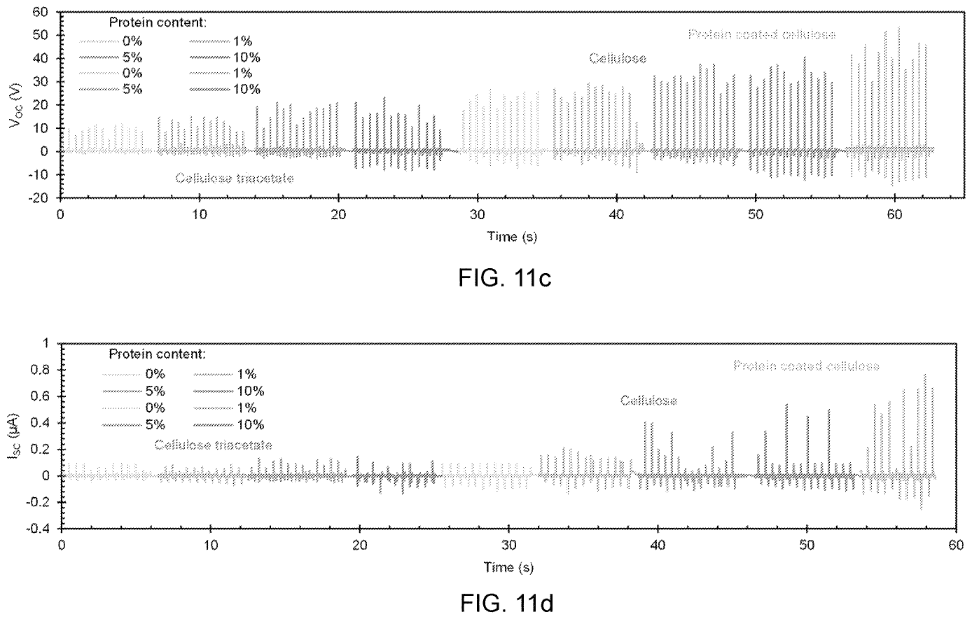

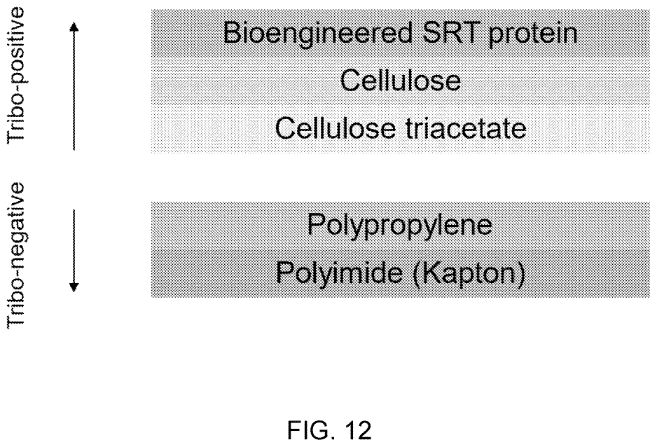

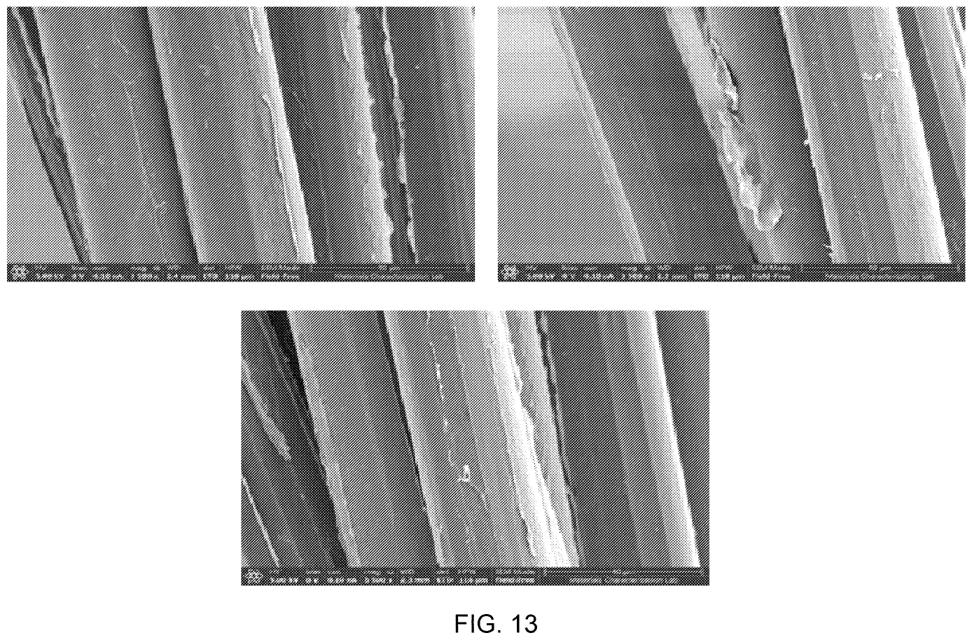

FIG. 4c shows the current and voltage measurements as a function of mechanical motion in time. The maximum recorded electrical outputs for protein films in the single and two-electrode configurations were 174 V and 4.82 μA, and 104 V and 1.12 μA, respectively. FIG. 11 shows voltage and current signals for cellulose fibers and cellulose triacetate. Cellulose fibers consistently outperform cellulose triacetate. According to FIG. 12, cellulose triacetate has a higher electron affinity than cellulose, making both materials tribologically positive. Notably, blending with protein significantly improved the triboelectric performance of both fibers. Triacetate and cellulose fibers showed a 72-108% and 49-57% increase in voltage when a protein content of 10% wt was added, as shown in FIGS. 4d and 4e. The current increases with more charges, indicating that protein has higher triboelectric strength than cellulose, inflating the electronegativity differences between the counter-layer materials. We also conducted an experiment to determine a maximum electrical output of cellulose fibers. We achieved this by coating the fibers with SRT protein using a dip coating method. This was designed to create a thin protein film on the surface of the fibers, resulting in a triboelectric output that primarily reflects protein characteristics. SEM micrographs of protein-coated pure cellulose fibers are shown in FIG. 13, where the protein deposits can be seen clearly. These fibers exhibit the highest voltage of all other fibers, i.e., 60.48 and 25.38 V·cm⁻² in single and two-electrode modes.

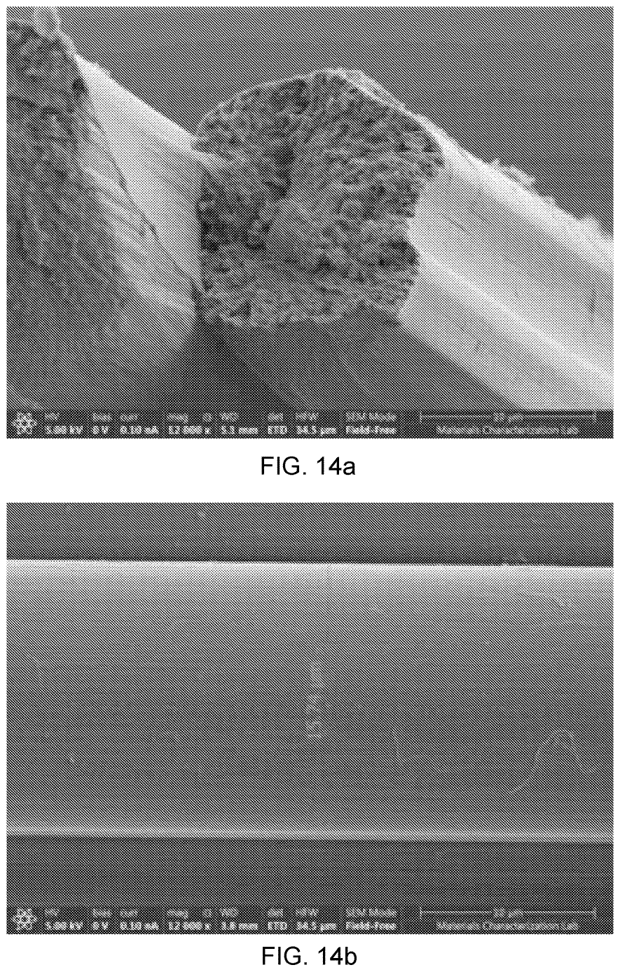

With the decrease in fiber diameter, the filter's filtration efficiency increases significantly. The disclosure demonstrates flexibility of the solution-spinning method by producing finer and more delicate fibers made of cellulose and protein. We were able to spin filaments with a diameter of approximately 15 μm, as shown in FIG. 14. Finer fibers exhibit greater triboelectric charge generation due to higher specific surface area.

FIG. 5 illustrates the normalized voltages and currents for devices from the literature (see Table S1 for references). The devices are segregated into categories, namely, polymer, cellulose, protein-based, and respirator filters. In single electrode mode, our cellulose-protein blend fibers surpass all other materials with a normalized voltage of 40.19 V·cm⁻². The voltage of cellulose fibers can be increased to 60.48 V·cm⁻² by coating them with our protein, twice the highest reported, as shown in Table S1. The percentage of protein in regenerated fibers can affect their triboelectric properties, as observed in FIG. 5. The two-electrode mode results show lower values than the single mode but confirm that our fibers perform better than the existing fibrous materials. Other proteinaceous fibrous materials such as silk, chitosan, and wool are not as effective as their normalized voltages lie under 9 V cm⁻². Unlike the current, without intending to be bound by any particular theory, it is considered that the maximum voltage (VOC) is the best indicator of accumulated charge, which also depends on electrode properties and device configuration. Cellulose-protein blend fibers exhibit the highest charge accumulation among all sustainable triboelectric materials discussed.

### Protein Recovery and Porous Biopolymer Fibers

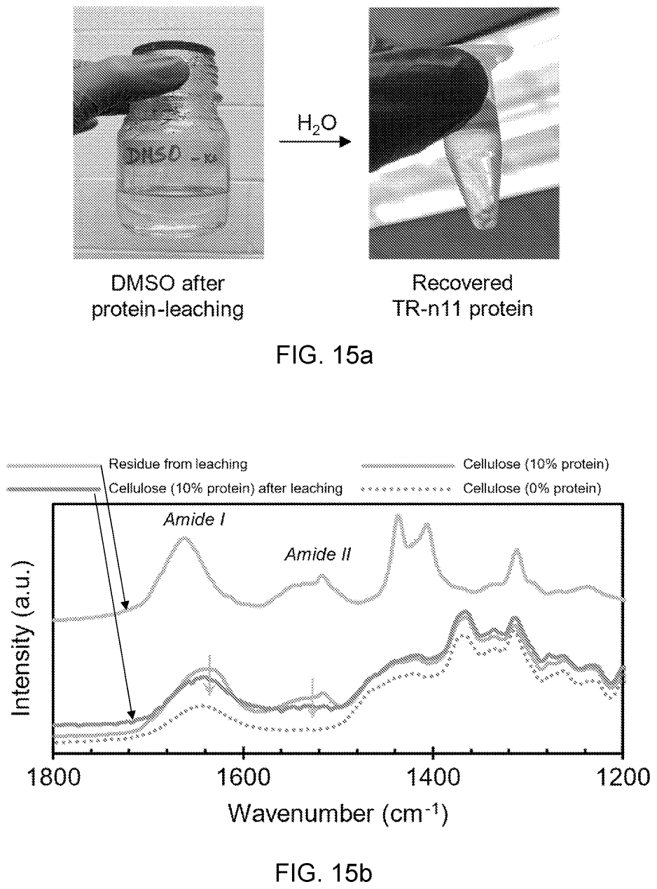

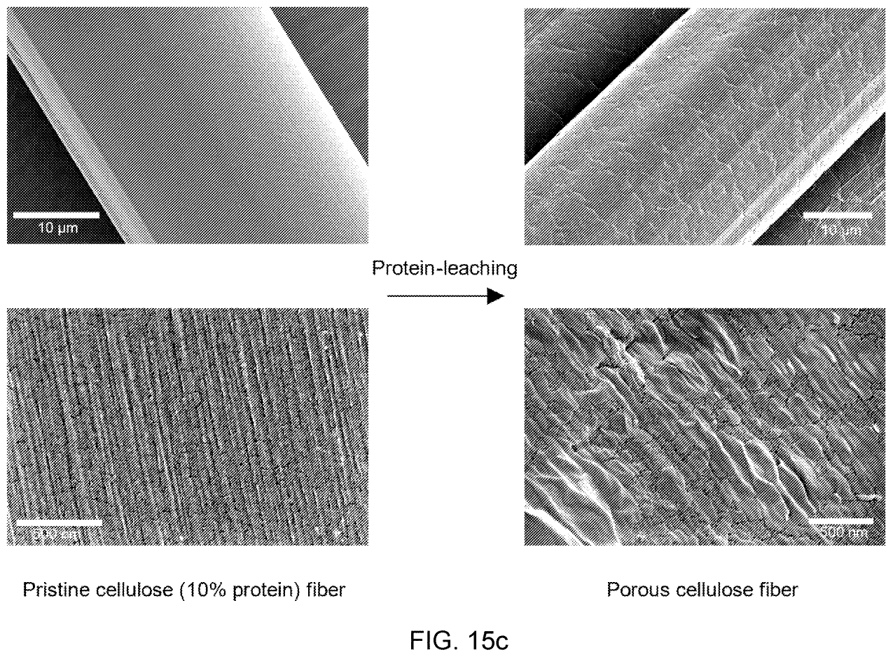

In the textile industry, fiber recycling is crucial for promoting sustainability and extending the lifespan of materials. We demonstrated the recyclability of protein in cellulose fibers by performing leaching experiments using dimethylsulfoxide (DMSO) as a non-limiting proof of concept. Proteins dissolve well in DMSO, while cellulose does not dissolve well in this organic solvent. Initially, fibers were kept in an oven at 60° C. for one hour to remove moisture. 215 mg of filaments were immersed in 50 ml of DMSO at 60° C. and were continuously stirred for a day. Following the DMSO treatment, the fibers were centrifuged to remove excess DMSO. The fibers were then washed and transferred to warm ultrapure water for 1 hr. Lastly, the fibers were dried in an oven at 60° C. for 3 hours and stored in a desiccator before characterization. The protein was separated from DMSO by adding excess ultrapure water after treating the filaments. FIG. 15a shows the residue extracted from fibers. FTIR spectra, as shown in FIG. 15b, confirmed protein extraction from fibers. DMSO-treated fibers show reduced Amide I and II band intensities, which are also apparent in the protein residue. Fibers underwent significant morphological changes during the treatment, as shown in FIG. 15c. Coalescence of surface regions was observed in optical images. We suggest that the swelling of cellulose in DMSO and water caused the coalescence of projected parts during drying. During the process of protein leaching, the fibers underwent a significant change that resulted in an increase in porosity. The diameter of treated fibers was higher by ≈2 μm, amounting to a net porosity of 10%.

It will be recognized from the foregoing examples that this disclosure provides in one aspect a method that can efficiently create fibers with exceptional triboelectric characteristics using sustainable biopolymers sourced from cellulose and biomanufactured proteins. These fibers can act as a substitute for plastics in various applications, including protective respirators or masks, and other devices as further described above. We demonstrated that the triboelectric voltage of regenerated fibers increased by 72-108% for cellulose and 49-57% for cellulose-triacetate, with a protein content of 10% wt. The application of biomanufactured protein coating on regenerated cellulose fibers significantly increases their triboelectric voltage, effectively doubling it. Fiber morphology plays a complex role in determining mechanical and triboelectric properties. We observed crystalline and amorphous regions in fiber morphology, which shape the fibers' overall mechanical properties, encompassing factors such as tensile strength, toughness, and resistance to deformation. Similarly, the triboelectric properties of the fibers were influenced by both the crystalline and amorphous sections. The specific surface characteristics, charge transfer traits, and overall composition of the protein collectively contribute to the triboelectric performance of the fibers. The disclosure accordingly demonstrates use of biomanufactured materials in reducing waste and driving technology towards a sustainable future.

### Example 3

This Example provides supplemental information that relates to the foregoing examples.

Scanning Electron Microscopy (SEM) microscopy in FIG. 6 provides us with enhanced views of these two materials at 1% and 5% protein content, allowing for closer inspection of the different compositions each contains. Examining fibers with varying protein content reveals a homogenous cross-section in their micrographs for cellulose fibers compared to triacetate counterparts.

SEM micrographs in FIG. 7 reveal similarities between cellulose fibers with and without protein content. At 10% protein, the morphology of as-spun fibers lacks any voids, which shows enhanced interaction between the cellulose and the proteins. The difference in the diameters of the two fiber types is due to differences in dope solid contents and spinning parameters. The diffusion kinetics of DMSO is faster than the ionic liquid¹. This results in the coagulation of the fiber surface faster than the fiber core. Since cellulose tends to absorb water and become plasticized, drawing resulted in a smoother surface for cellulose than triacetate. A smooth surface undergoes specular reflection, enhancing the aesthetics of the fiber. Compared to the compact structure of cellulose fibers, triacetate fibers contained nanopores. It has been noted that the high diffusion rate of DMSO creates porous fibrils. The rapid escape of solvent from the fiber leads to faster coagulation of the dope, such that the in-line tension cannot stretch the protofibril adequately to induce a dense microstructure. Moreover. SEM micrographs of fiber cross-sections do not show phase separation of the protein. No protein precipitates in the coagulation bath were seen either.

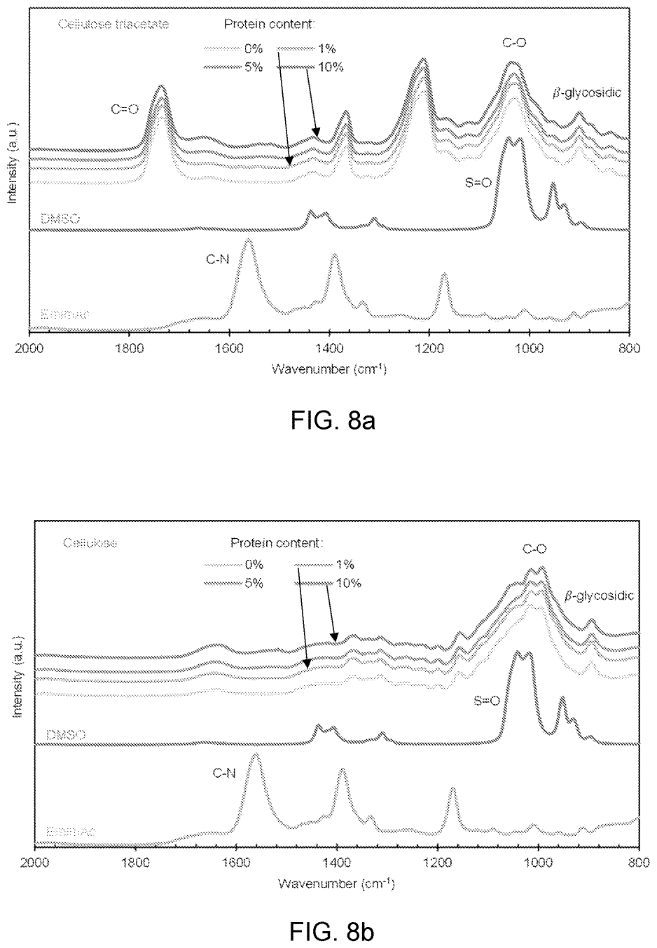

Fourier Transform Infrared Spectroscopy (FTIR) analysis of cellulose triacetate and cellulose fibers and related solvents in FIG. 8 was conducted to investigate chemical composition. The findings show that the fibers lack any solvent used in the wet-spinning of the fibers and confirm the existence of protein in the fibers. In addition, FIG. 9 displays the deconvoluted spectra for the SRTs in cellulose (a) and cellulose triacetate (b), along with the fitted set of secondary structure bands. FIG. 10 shows the schematic of the triboelectric nanogenerator in both single electrode (a) and dual-electrode configurations (b). By combining protein film with two electrodes, a triboelectric nanogenerator is constructed that utilizes mechanical energy to generate electricity.

In FIG. 11, cellulosic fibers coated with our proteins have been assessed as potential devices for triboelectric energy harvesting. Our analysis revealed that their output can reach an impressive maximum when configured in either single or two-electrode systems, potentially opening new avenues of exploration in energy harvesting. Both open circuit voltages (VOC) and short-circuiting currents (ISC) were tracked to assess performance levels across the context range studied here.

FIG. 12 shows a schematic, which offers an insight into how the materials employed in this study interact electrostatically, as illustrated by their respective positions on the triboelectric series.

Protein deposits have been observed to coat the surfaces of cellulose fibers, in FIG. 13, when viewed under a SEM micrograph, providing an interesting insight into uniform structure on this scale.

FIG. 14 shows distinct views from cross-section as well as lateral surface visuals. These SEM micrographs revealed that wood cellulose fibers can be drawn into finer diameter of 15 microns. While mechanisms including impaction, diffusion, and interception are responsible for most of the filtration efficiency of respirators (83-92%)², extending the efficiency further by engineering the filter architecture alone is futile. This restriction is overcome by introducing surface charges on constituent fibers, allowing particulate capture by the electrostatic mechanism. N95 respirators, commonly based on polypropylene electret, portray filtration efficiencies of over 95%^{3,4}. However, the standard methods of charging fibers, i.e., induction and corona discharge, require hundreds of kilovolts, making the processes extraordinarily energy-intensive and hazardous⁵. The industry can realize the triboelectric effect through carding⁶.

Herein, by incorporating unique bioengineered SRT protein, the triboelectric performance of cellulosic fibers was enhanced significantly. Since the filtration efficiency of filters is directly correlated to the surface charge density of fibers, the functionalization of fibers by proteins is highly favorable. Moreover, by modifying the amount of protein in the blend, the triboelectric properties of the fibers can be fine-tuned. Yim et al. studied three N95 and four KN95 commercial respirators. While the surface charge density on KN95 was lower, their filtration efficiency was comparable to that of NIOSH-approved N95 masks. Therefore, if the filter architecture is optimized appropriately per the mechanical mechanisms, it can portray high performance even with low surface charge density. The general construction of a respirator comprises an outer protective, filter (middle), and inner protective polymeric fibrous layers. While the filter layer is the main functional element, the protective layer provides structural support and safety. Cellulose esters are promising substitutes for polymers for protective coatings. They are desirable from the viewpoints of their mechanical properties and high hydrophobic nature¹³. Being hydrophobic, the cellulosic layers would keep moisture from the filter layer, preventing extensive charge decay in humid conditions. Moreover, with the lowering of fiber diameter, the filtration efficiency of the filter improves exponentially. We demonstrate the versatility of the wet spinning process by spinning finer and more delicate cellulose-protein fibers. High molecular weight cellulose was spun into filaments of diameter ˜15 μm (FIG. 14), approximately one-half of the cellulose blend fibers. Finer fibers also exhibit more substantial triboelectric charge generation due to higher specific surface area.

Tables S1 and S2 show a list of literature referred in the main text and surface areas of fibers used in this study, respectively. The maxima of output voltages of polymer and cellulose-based devices are similar, with the materials being ZnO-doped polyvinylidene fluoride with nylon and nitrocellulose membranes with crimped paper, respectively (Table S1). Moreover, several materials are often integrated to achieve considerable performance (as high as 5), including Ag nanowires, BaTiO3, MXene, fluorinated polymers, silanes, carbon nanotubes, graphene oxide, and many more. This disclosure demonstrates performance enhancement by incorporating a single protein variant, thus, curbing material diversity. Additionally, Shen et al. discussed polypropylene-based devices. They used Aluminum as the positive layer. The maximum voltage attained at 10 N normal force was approx. 20 V. In addition to fabrics, they tested commercial surgical masks and N95 filter layers. The max voltages for the mask and N95 were 10 and 13 V, respectively. Compared to the presently described biopolymer-protein fibers, the triboelectric performance of polypropylene was inferior.

TABLE S1

| Category | S. No. | Materials* |
|---|---|---|
| Polymer | 1. | PP, Al, Cu, N95 filter, surgical mask |
| based | 2. | TPU, PP, Ni-coated fabric |
| | 3. | PA6, Cu, cotton, dacron |
| | 4. | PLA, Cu, TPU/Au, PVDF |
| | 5. | PTFE, PA6, Graphene |
| | 6. | PTFE, PA6, Ag |
| | 7. | SS/PET, Si-rubber |
| | 8. | PET, Ni, parylene |
| | 9. | PTFE, Ag |
| | 10. | PI, PU, PDMS, Al, carbon fabric |
| | 11. | PET, PVDF NFs, PTFE NPs, CFab |
| | 12. | PET, Si-rubber, Ni |
| | 13. | Al, PA6, PVDF, CFab, |
| | 14. | PA6, PET, Ag |
| | 15. | PDMS, PVDF-HFP, Cu, Kapton |
| | 16. | PU, SS, PET |
| | 17. | Si-rubber, PEDOT:PSS, Al, PTFE |
| | 18. | AgNPs, PVDF, PET, PAN, TPU |
| | 19. | CFab, PET |
| | 20. | Ag, chinlon, PDMS, SS |
| | 21. | Ni/PET, PDMS-CFab |
| | 22. | PA6, PTFE, Au, PU |
| | 23. | Si, PET, CFab |
| | 24. | PDMS, Al NP coated fabric |
| | 25. | Si, SS, skin (as electrode) |
| | 26. | PVA/H₃PO₄, CNT/WPU, |
| | 27. | PET, PVDF, PA6, silk, Al |
| | 28. | PVDF, PA6, ZnO, Al |
| Protein | 29. | CMCS, CMC-Na, Au |
| based | 30. | Silk, Si-rubber, CFab |
| | 31. | Si, PA6, CNT, skin (as electrode) |
| | 32. | Cotton, Ni, wool, skin (as electrode) |
| | 33. | Si, Cu, liquid metal, PTFE |
| | 34. | Chitosan, silk, PTFE, Al |
| | 35. | Silk, SS, PTFE |
| Cellulose | 36. | PS, PES, CA, Cu, PMMA, PI |
| based | 37. | EC/PA6, PVDF/MXene, Cu |
| | 38. | CEL, CNF, Ag, FEP |
| | 39. | Paper, PVDF |
| | 40. | CNF, AgNW |
| | 41. | PET, Ag, PDMS, CNF |
| | 42. | Cotton, CFab, PA6 |
| | 43. | Paper, ITO, PDMS/PVDF, FEP |
| | 44. | AgNW, BaTiO₃, bacterial-CEL, PTFE, Al |
| | 45. | Paper, nitro-CEL, Cu |
| | 46. | Paper, PTFE, graphite |
| | 47. | Cotton, PTFE, CFab, PET |
| | 48. | Cu, paper, nitro-CEL |
| | 49. | FEP, ITO, PET, CNF |
| | 50. | Paper, PCL/GO, Au |
| | 51. | CEL, nitro-CEL, pyrrole-CEL |
| | 52. | Cotton, PET, Ni, parylene |
| | 53. | Wood, PTFE, Cu |
| | 54. | Al, paper, PET, PVDF, Ag, PI |
| | 55. | Acrylic, PET, ITO, FEP, CNC |
| | 56. | CNF, methyl-CNF, nitro-CNF, FEP-CNF, Cu |
| | 57. | Cu, PTFE, lignin |
| | 58. | CEL, CNF, PTFE, Al |
| | 59. | MCC, HCOENPs, PET, Au, CFab |

TABLE S2
Effective surface areas (in cm²) of fiber-based devices.

| | | Protein content (%) | | | |
|---|---|---|---|---|---|
| Fiber type | Device type | 0 | 1 | 5 | 10 |
| Cellulose triacetate | Single electrode | 1.92 | 2.19 | 2.17 | 2.28 |
| | Two electrodes | 2.19 | 2.20 | 1.90 | 2.03 |
| Cellulose | Single electrode | 2.13 | 2.28 | 2.15 | 2.07 |
| | Two electrodes | 2.09 | 2.26 | 2.29 | 2.11 |

| | | |
|---|---|---|
| Protein-coated | Single electrode | 2.05 |
| cellulose | Two electrodes | 2.08 |

Other embodiments of the disclosure will be apparent to those skilled in the art from consideration of the specification and practice of the disclosure disclosed herein. It is intended that the specification and examples be considered as exemplary only.

1. A composition comprising a polysaccharide that is optionally a cellulosic material, and one or more polypeptides which comprise alternating repeats of crystallite-forming subsequences and amorphous subsequences, and wherein the composition is capable of exhibiting a triboelectric charge.

2. The composition of claim 1, wherein the crystallite-forming subsequences comprise β-sheets.

3. The composition of claim 1, wherein the cellulosic material is present and is selected from the group consisting of cellulose, cellulose triacetate, methylcellulose, hydroxypropyl methylcellulose, and a combination thereof.

4. The composition of claim 1, wherein the one or more polypeptides comprise 2 to 20 repeats of the crystallite-forming subsequences.

5. The composition of claim 1, wherein the crystallite-forming subsequences comprise or consist of the sequence AAASVSTVHHP (SEQ ID NO:1).

6. The composition of claim 5, wherein the amorphous subsequence comprises from 10 to 60 amino acids

7. The composition of claim 6, wherein the amorphous subsequences comprise or consist of the sequence YGYGGLYGGLYGGLGYGP (SEQ ID NO:2).

8. The composition of claim 1, wherein the composition is formed as a fiber.

9. The composition of claim 8, wherein the one or more polypeptides comprise approximately 10% of the weight of the fiber.

10. A plurality of fibers of claim 9.

11. A filter comprising a plurality of fibers of claim 10.

12. The filter of claim 11, wherein the filter is a component of a respirator.

13. A method of making a composition of claim 8, the method comprising solution spinning a composition comprising a cellulosic material and one or more polypeptides which comprise alternating repeats of crystallite-forming subsequences and amorphous subsequences to obtain a fiber comprising or consisting essentially of the cellulosic material and the one or more polypeptides.

14. The method of claim 13, wherein the cellulosic material is selected from the group consisting of cellulose, cellulose triacetate, methylcellulose, hydroxypropyl methylcellulose, and a combination thereof.

15. The method of claim 14, wherein the one or more polypeptides comprise 2 to 20 repeats of the crystallite-forming subsequences.

16. The method of claim 15, wherein the crystallite-forming subsequences comprise or consist of the sequence AAASVSTVHHP (SEQ ID NO:1).

17. The method of claim 16, wherein the amorphous subsequence comprises from 10 to 60 amino acids

18. The method of claim 17, wherein the amorphous subsequences comprise or consist of the sequence YGYGGLYGGLYGGLGYGP (SEQ ID NO:2).

19. A method comprising passing air through a filter of claim 11, wherein a triboelectric charge is formed in the filter such that particulate matter in the air is trapped on or within the filter.

20. The method of claim 19, wherein the fiber is a component of a respirator.

21. An article of manufacture comprising a filter of claim 11.

22. The article of manufacture of claim 21, wherein the filter is present in a reusable or disposable cartridge.

23. A system comprising a filter of claim 11, wherein the system comprises a conduit in fluid communication with the filter and is configured such that air passing through the filter immobilizes particular matter in the air on or within the filter.


SEQUENCE LISTING (2 sequences)

SEQ ID NO: 1 - protein, 11 aa, synthetic construct
AAASVSTVHHP

SEQ ID NO: 2 - protein, 18 aa, synthetic construct
YGYGGLYGGLYGGLGYGP